(19) United States
(12) Patent Application Publication
(10) Pub. No.: US 2025/0242144 A1
(43) Pub. Date: Jul. 31, 2025

(54) CATHETERS WITH IMPROVED TORQUE TRANSMISSION CAPABILITIES

(71) Applicant: ABIOMED, Inc., Danvers, MA (US)

(72) Inventor: Alexander Ship, Danvers, MA (US)

(73) Assignee: ABIOMED, Inc., Danvers, MA (US)

(21) Appl. No.: 19/037,108

(22) Filed: Jan. 25, 2025

Related U.S. Application Data
(60) Provisional application No. 63/742,671, filed on Jan. 7, 2025.
(60) Provisional application No. 63/726,997, filed on Dec. 2, 2024.
(60) Provisional application No. 63/625,779, filed on Jan. 26, 2024.

(51) Int. Cl. A61M 60/174; A61M 25/00; A61M 60/226; A61M 60/411

(52) U.S. Cl. CPC A61M 60/174; A61M 25/0026; A61M 25/0053; A61M 60/226; A61M 60/411

(57) ABSTRACT

Catheters for catheter assemblies, such as, intracardiac blood pump assemblies are provided with improved torque transmission capabilities.

### CROSS REFERENCE TO RELATED APPLICATIONS

This application claims the priority of and benefit from U.S. Provisional Application No. 63/625,779, filed Jan. 26, 2024, U.S. Provisional Application No. 63/726,997, filed Dec. 2, 2024, and U.S. Provisional Application No. 63/742,671, filed Jan. 7, 2025, each of which is incorporated by reference herein.

### TECHNICAL FIELD

The present technology relates to catheters for catheter assemblies, such as, intracardiac blood pump assemblies, and in particular to catheters with improved torque transmission capabilities.

### BACKGROUND

Catheter assemblies, such as, intracardiac blood pump assemblies can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

Catheter assemblies, such as, intracardiac blood pump assemblies can be introduced into the heart by a catheterization procedure. For example, with respect to an intracardiac blood pump assembly inserted into the left heart, an introducer sheath assembly may be inserted into the femoral artery through an arteriotomy to gain access to the artery and create an insertion path. A placement guidewire can be advanced into the artery along the insertion path. After the guidewire has been inserted into the artery, the pump assembly can be advanced over the guidewire and into the patient. Alternatively, the pump assembly can be inserted directly into the artery without a guidewire. The blood pump of the assembly can be inserted via a catheterization procedure through the femoral artery, into the ascending aorta, across the aortic valve and into the left ventricle. When deployed in the left heart, the pump assembly pulls blood from the left ventricle and expels blood into the ascending aorta.

### BRIEF SUMMARY

The present technology relates to catheters for catheter assemblies, such as, intracardiac blood pump assemblies, and in particular to catheters with improved torque transmission capabilities.

In one aspect of the present technology, a catheter for a catheter assembly is provided. The catheter comprises an elongate tubular body. The elongate tubular body comprises a proximal end and a distal end, at least one lumen extending from the proximal end to the distal end along a longitudinal axis, a layer made of a first material, a first portion having a first length, a second portion having a second length, wherein the first portion is proximal of the second portion, and a reinforcement structure embedded in the layer, wherein the reinforcement structure is configured such that the second portion of the elongate tubular body transmits a larger proportion of externally applied torque over the second length than the first portion transmits over the first length.

In some aspects of the catheter, the reinforcement structure is made of a second material.

In some aspects of the catheter, the first material is a polymer.

In some aspects of the catheter, the first material is a polyurethane.

In some aspects of the catheter, the second material is a metal alloy.

In some aspects of the catheter, the second material is nitinol.

In some aspects of the catheter, the reinforcement structure comprises a first portion that extends from a first end to a second end of the first portion of the elongate tubular body, and the first portion of the reinforcement structure is configured as a coil.

In some aspects of the catheter, the reinforcement structure comprises a second portion that extends from a first end to a second end of the second portion of the elongate tubular body, and the second portion of the reinforcement structure is laser cut according to a predetermined pattern.

In some aspects of the catheter, the predetermined pattern comprises apertures in the reinforcement structure that are arranged such that a first spiral torque path and a second spiral torque path are formed by material of the reinforcement structure between the apertures.

In some aspects of the catheter, the first spiral torque path and second spiral torque path are wound about the longitudinal axis in opposing directions.

In some aspects of the catheter, the apertures are each diamond-shaped.

In some aspects of the catheter, the predetermined pattern comprises a lattice structure.

In some aspects of the catheter, the reinforcement structure has an elongate tubular shape.

In some aspects of the catheter, the reinforcement structure is coaxial with the elongate tubular body.

In some aspects of the catheter, the elongate tubular body further comprises an intermediate portion disposed between the first portion and the second portion and the reinforcement structure further comprises an intermediate portion that extends from a first end to a second end of the intermediate portion of the elongate tubular body.

In some aspects of the catheter, the elongate tubular body is formed by reflowing the first material onto the reinforcement structure and the intermediate portion of the reinforcement structure includes one or more features configured to promote mechanical interlocking between the one or more features and the layer when the first material is reflowed onto the reinforcement structure.

In one aspect of the present technology, an intracardiac blood pump assembly is provided. The intracardiac blood pump assembly comprises: a blood pump comprising a proximal portion and a distal portion; and a catheter comprising an elongate tubular body. The elongate tubular body comprises: a proximal end and a distal end, wherein the distal end of the elongate tubular body is coupled to the proximal portion of the blood pump, at least one lumen extending from the proximal end to the distal end along a longitudinal axis, a layer made of a first material, a first portion having a first length, a second portion having a second length, wherein the first portion is proximal of the second portion, and a reinforcement structure embedded in the layer, wherein the reinforcement structure is configured such that the second portion of the elongate tubular body transmits a larger proportion of externally applied torque over the second length than the first portion transmits over the first length.

In some aspects of the intracardiac blood pump assembly, the reinforcement structure comprises a first portion that extends from a first end to a second end of the first portion of the elongate tubular body, and the first portion of the reinforcement structure is configured as a coil.

In some aspects of the intracardiac blood pump assembly, the reinforcement structure comprises a second portion that extends from a first end to a second end of the second portion of the elongate tubular body, and the second portion of the reinforcement structure is laser cut according to a predetermined pattern.

In some aspects of the intracardiac blood pump assembly, the predetermined pattern comprises apertures in the reinforcement structure that are arranged such that a first spiral torque path and a second spiral torque path are formed by material of the reinforcement structure between the apertures, wherein the first spiral torque path and second spiral torque path are wound about the longitudinal axis in opposing directions.

### BRIEF DESCRIPTION OF THE DRAWINGS

FIG. 1A illustrates an exemplary blood pump portion of an intracardiac blood pump assembly for left heart support in accordance with aspects of the present technology.

FIGS. 1B and 1C illustrate additional components of the exemplary intracardiac blood pump assembly of FIG. 1A in accordance with aspects of the present technology.

FIG. 1D illustrates the blood pump portion of the exemplary intracardiac blood pump assembly of FIG. 1A inserted into the patient in accordance with aspects of the present technology.

FIG. 2 illustrates a portion of the exemplary intracardiac blood pump assembly of FIG. 1A including a securement device in accordance with aspects of the present technology.

FIG. 3 illustrates a catheter and a blood pump of an intracardiac blood pump assembly in accordance with aspects of the present technology.

FIG. 4 is a cross-sectional view of the catheter of FIG. 3 along line A-A and illustrates a reinforcement structure of the catheter in accordance with aspects of the present technology.

FIG. 5 illustrates a portion of the reinforcement structure of FIG. 4 in accordance with aspects of the present technology.

### DETAILED DESCRIPTION

Aspects of the present technology are described in detail with reference to the drawing figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

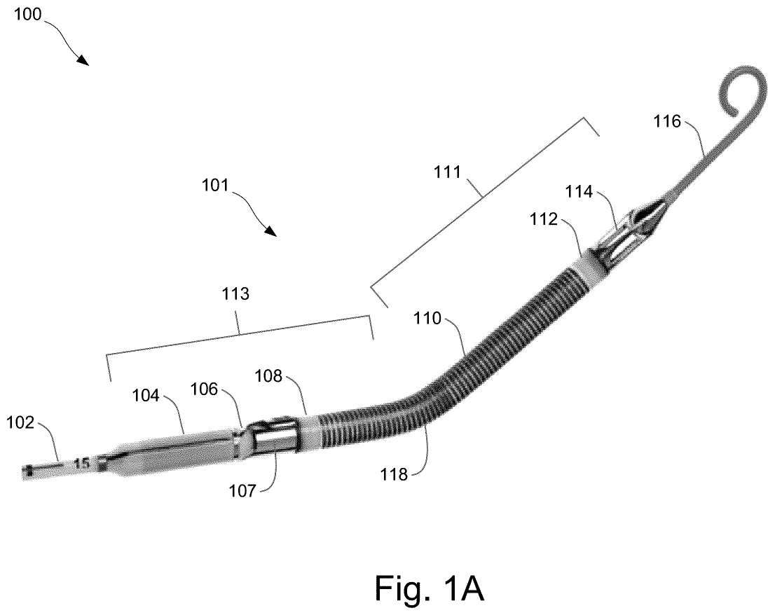

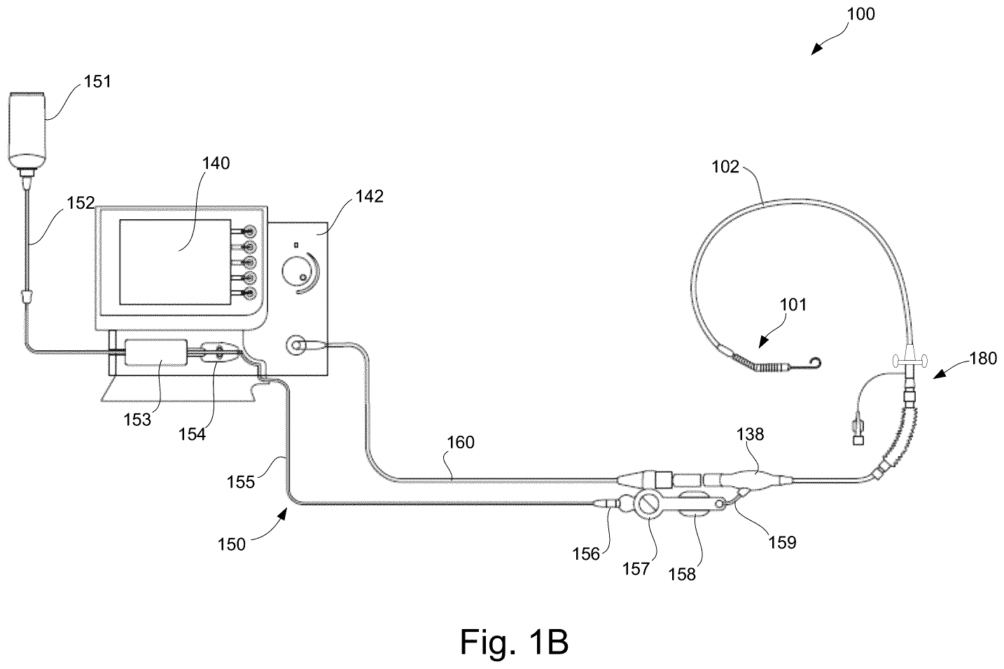

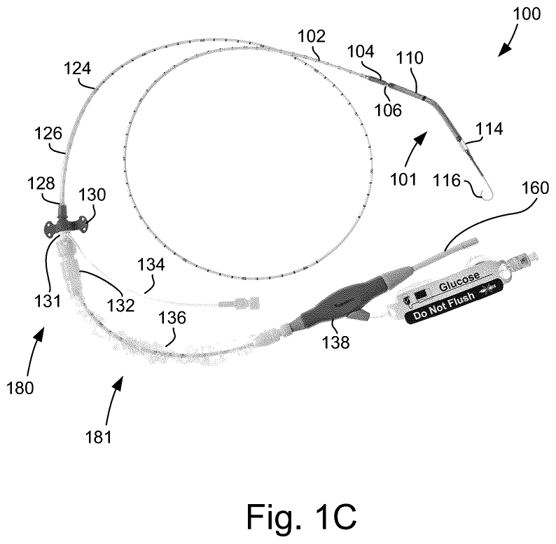

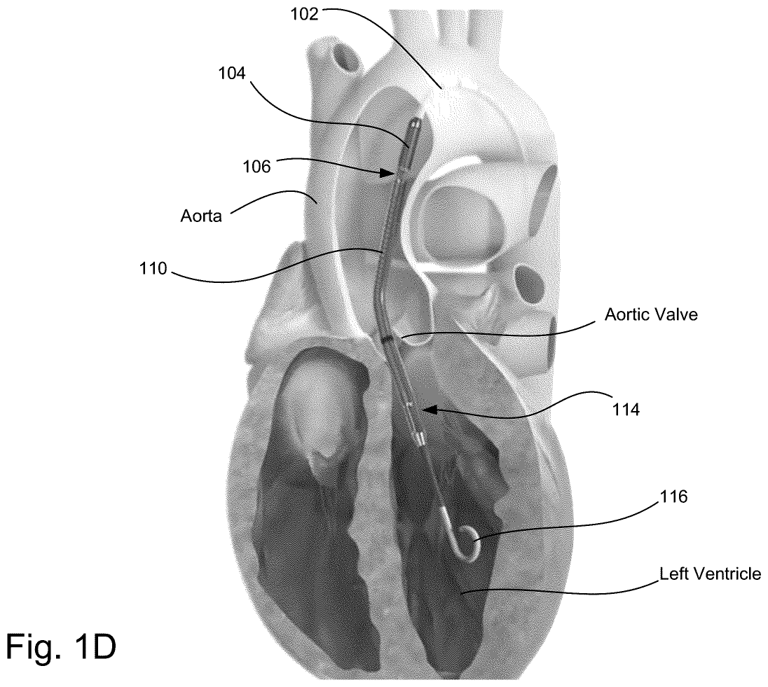

FIG. 1A depicts a blood pump portion 101 of an exemplary intracardiac blood pump assembly 100 adapted for left heart support, in accordance with aspects of the present technology. As shown in FIG. 1A, an intracardiac blood pump assembly 100 adapted for left heart support may include an elongate catheter 102, a motor housing 104, a cannula 110, a blood flow inlet 114 arranged at or near the distal end 112 of the cannula 110, a blood flow outlet 106 arranged at or near the proximal end 108 of the cannula 110, and an optional atraumatic extension 116 arranged at the distal end of the blood inflow cage 114. In one aspect, the inlet 114 is configured as a blood inflow cage and the outlet 106 is configured as a blood outflow cage. The motor 104, cannula 110, blood inflow cage 114, blood outflow cage 106 form a blood pump portion 101 of assembly 100. The blood pump portion 101 has a first portion 111 and a second portion 113. Portion 113 is proximal to portion 111. In one aspect, pre-formed bend 118 is disposed between the first portion 111 and the second portion 113.

In some aspects of the present technology, motor housing 104 houses a motor (not shown) that is configured to rotatably drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a pump housing 107 coupled to the proximal end 108 of the cannula 110. In some aspects of the technology, rather than the impeller being driven by an onboard motor in motor housing 104, the impeller may instead be coupled to an elongate drive shaft (or drive cable) which is driven by a motor located external to the patient.

Catheter 102 may house electrical lines coupling the motor in motor housing 104 to one or more electrical controllers and/or sensors. Alternatively, where the impeller is driven by an external motor, an elongate drive shaft may pass through catheter 102. Catheter 102 may also include a purge fluid conduit, a lumen configured to receive a guidewire, one or more optical fibers (e.g., for sensing pressure), etc.

The blood inflow cage 114 may include one or more apertures or openings configured to allow blood to be drawn into cannula 110 when the motor in motor housing 104 is operating. Likewise, blood outflow cage 106 may include one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, as shown in the exemplary arrangement of FIG. 1A, the cannula 110 may have a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

Atraumatic extension 116 may assist with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, etc. Atraumatic extension 116 may also have sections where at least one section has a stiffness that is different from the stiffness of another section. For example, atraumatic extension 116 may include a proximal section that is sufficiently stiff to resist buckling when subjected to the typical forces applied to the atraumatic tip when the pump is deployed, thereby keeping the blood inflow cage 114 in the desired location. The atraumatic extension 116 may also include a distal section that is softer and has a lower stiffness than the proximal section, thereby providing an atraumatic tip that may safely come into contact with a wall of the patient's heart when deployed and may be compatible with guidewire loading of the blood pump. In such a case, the proximal and distal sections of the atraumatic extension 116 may be composed of different materials, or may be composed of the same material with the proximal and distal sections being treated so that one section has a stiffness that is different from the stiffness of another section.

Notwithstanding the foregoing, as mentioned above, atraumatic extension 116 is an optional structure. In that regard, the present technology may also be used with intracardiac blood pump assemblies and other intracardiac devices that include different types of extensions than those specifically described herein. These include extensions with different shapes than those described, extensions made of different materials than those described, extensions with different features than those described, etc. Likewise, the present technology may be used with intracardiac blood pump assemblies and other intracardiac devices that do not have a distal extension of any kind.

As shown in FIG. 1A, the distal end of catheter 102 is coupled the proximal end of proximal portion 113 (e.g., to motor housing 104). The proximal portion of catheter 102 is further coupled to additional components of blood pump assembly 100, as shown in FIG. 1B.

In this regard, as shown in FIG. 1B, in addition to blood pump 101 and catheter 102, the blood pump assembly 100 may further include a purging device or assembly 150, a controller 142 (e.g., an Automated Impella Controller® from Abiomed, Inc., Danvers, MA), a display 140, a connector cable 160, a plug 138, and a repositioning unit 180. In some aspects, controller 142 includes display 140. Controller 142 comprises one or more processors. Controller 142 monitors and controls blood pump 101. During operation, purging device 150 delivers a purge fluid to blood pump 101 through catheter tube 102 to prevent blood from entering the motor (not shown) within motor housing 104. In some implementations, the purge fluid comprises a dextrose solution (e.g., 5% dextrose in water with 25 or 50 IU/mL of heparin). Connector cable 160 may provide electrical and/or optical connection(s) between blood pump 101 and controller 142. Plug 138 connects catheter tube 102, purging device 150, and connector cable 160. In some embodiments, plug 138 includes a memory for storing operating parameters in case the patient needs to be transferred to another controller 142. As will be described in greater detail below, repositioning unit 180 may be used as a tool to (position and) reposition blood pump 101 within a patient.

As shown, purging device 150 comprises a reservoir 151, purge fluid supply line 152, a purge cassette 153, a purge disc 154, purge tubing 155, a check valve 156, a pressure reservoir 157, an infusion filter 158, and a sidearm 159. Reservoir 151 may, for example, be a bag or a bottle. A purge fluid is stored in reservoir 151. A purge fluid spike at the end of purge fluid supply line 152 may be used to puncture reservoir 151 and connect the purge fluid in reservoir 151 to purge fluid supply line 152. Purge fluid supply line 152 carries the purge fluid from reservoir 151 to purge cassette 153. Purge tubing 155 carries the purge fluid from purge cassette 153 to blood pump 101.

Purge cassette 153 controls how the purge fluid in reservoir 151 is delivered to blood pump 101 and the flow path of the purge fluid from reservoir 151 to blood pump 101. For example, purge cassette 153 may include one or more valves (e.g., purge path diverters) for controlling a pressure and/or flow rate of the purge fluid. In addition to containing the components for delivering the purge fluid, purge cassette 153 also maintains the pressure barrier between the blood and the motor of blood pump 101 to prevent blood from entering the motor. Purge cassette 153 may contain a rack and pinion which is attached to a piston. Purge disc 154 includes one or more measuring device(s), such as pressure sensors (e.g., a pressure-sensing diaphragm) for measuring purge pressure of the purge fluid at blood pump 101. Controller 142 is connected to purge cassette 153 and purge disc 154. Purge disc 154 transmits pressure to controller 142 based on the purge pressure in purge tubing 155. A sensor in controller 142 measures the pressure so that it can be displayed on screen 140. Controller 142 may include a stepper motor. A tic (or step) represents stepper motor positions in units of microsteps, which are also called pulses. In some embodiments, a purge pressure/purge flow curve algorithm is deployed by the controller 142 in conjunction with the number of steps/minute of the stepper motor and pressure measurements by purge disc 154 in order to calculate the corresponding purge flow rate.

As described above, purge tubing 155 provides a fluidic connection passing through purge cassette 153 to blood pump 101. In some embodiments, a Luer-lock connector is also provided, which facilitates a continuous fluid path or purge fluid path. A Luer-lock connector connects purge tubing 155 to a check valve 156 (which may be a Luer-lock) in the purge fluid path from the reservoir 151 to the blood pump 101. Pressure reservoir 157 provides additional filling volume during a purge fluid change. In some embodiments, pressure reservoir 157 includes a flexible rubber diaphragm that provides the additional filling volume by means of an expansion chamber. Infusion filter 158 helps prevent bacterial contamination and air from entering catheter tube 102. Sidearm 159 provides a fluidic connection between infusion filter 158 and plug 138.

During operation, controller 142 receives measurements from purge disc 154 and controls the stepper motor's number of ticks to control the purge pressure. In some embodiments, during operation, purge cassette 153 is placed in controller 142 and connected with the purge line to blood pump 101. As noted above, controller 142 controls and measures purge pressure and calculates purge flow rate via purge cassette 153 and/or purge disc 154. Controller 142 may also control the purge fluid supply. During operation, after exiting purging device 150 through sidearm 159, the purge fluid is channeled through purge lumens (not shown) within plug 138 and catheter tube 102. Sensor cables (not shown) within catheter tube 102, connector cable 160, and plug 138 provide an electrical connection between purge disc 154 and controller 142. Motor cables (not shown) within catheter tube 102, connector cable 160, and plug 138 provide an electrical connection between the motor within motor housing 104 and controller 142. During operation, controller 142 receives measurements from purge disc 154 through the sensor cables and controls the electrical power delivered to the motor within motor housing 104 through the motor cables. By controlling the power delivered to the motor within motor housing 104, controller 142 may control the speed of the motor within motor housing 104. In some embodiments, controller 142 includes safety features to prevent air from entering purge tubing 155. Controller 142 may include (or be in communication with) circuitry for monitoring the motor current for drops in current indicating air in the line. Controller 142 may include or be configured to generate warning sounds, lights or indicators to alert an operator of certain detected conditions, such as, but no limited to, pump position, suction events at the inlet, and disconnects or breaks in purge tubing 155 which may result in the introduction of air to the line.

In some aspects, assembly 100 may include one or more sensors or measurements devices configured to communicate with controller 142 to provide information associated with the operation of assembly 100 or a patient. In one aspect, assembly 100 may include an optical fiber (disposed through one or more of cable 160, plug 138, and catheter 102) that forms a pressure sensitive surface at its distal end. The pressure sensitive surface forms a pressure sensor which may be added to blood pump 101 near its inlet area 114. The pressure sensor is configured to measure a left ventricular blood pressure. Assembly 100 may implement multiple such pressure sensors (or other sensor types) at different locations (e.g., on or in pump 101, catheter 102, plug 138, etc.) throughout assembly 100 and controller 142 may be configured to perform different steps based on the information received for the sensors. In some aspects, additional sensor cables may be disposed within catheter tube 102, connector cable 160, and plug 138 to provide an electrical and/or optical connection between the one or more additional measuring devices and controller 142. As yet another example, one or more components of blood pump assembly 100 may be separated. For example, display 140 may be incorporated into another device in communication with controller 142 (e.g., wirelessly or through one or more electrical cables).

Display 140 is controllable by controller 142 to display useful information to the user of the blood pump assembly 100. For example, display 140 may be controlled to display many different types of information such as the characteristics of the blood pump assembly (e.g., blood pump type, serial number, software version, etc.), operation of the blood pump assembly (e.g., present blood pump speed (performance) setting, blood pump flow measurements, purging device measurements, a status indicator, sensor measurements, blood pump position detections and indications, etc.). Some of this information may be obtained from purge disc 154 or any of the other sensors described above or that may be used with assembly 100. Display 140 can also provide notifications to the user. For example, a notification may serve as an alert and include a statement describing the cause of the alert. In some embodiments, display 140 may be a touchscreen and a user may switch between screens by tapping button labels on display 140. In some embodiments, a user may use a separate input device, such as a mouse or a keyboard, to switch between screens.

As described above, assembly 100 may include a repositioning unit 180 for repositioning the blood pump 101 within the patient. For example, FIG. 1C shows an exemplary implementation of blood pump assembly 100 with a repositioning unit or assembly 180, in accordance with aspects of the present technology.

In one aspect, the repositioning unit 180 may include repositioning sheath 126, fixation device or butterfly 130, hemostasis valve 131, securement device 132, and protective sleeve 136. In this aspect, between handle 138 and securement device 132, the catheter 102 is enclosed within a protective sleeve 136. Protective sleeve 136 may be configured to prevent contamination of catheter 102 as it is advanced in the distal direction for insertion into the patient's vasculature. Protective sleeve 136 may be comprised of any suitable material, and may be secured at its proximal and distal ends in any suitable manner. The distal end of protective sleeve 136 may be coupled to securement device 132. In the example of FIG. 1C, securement device 132 is coupled at its distal end to a hemostasis valve 131, which in turn is attached to butterfly 130. Hemostasis valve 131 may be integrated with butterfly 130, or may be removably coupled thereto. In addition, hemostasis valve 131 and securement device 132 may be incorporated into a single unit. Butterfly 130 is coupled at its distal end to the proximal end 128 of repositioning sheath 126. The distal end of repositioning sheath 126 is indicated with reference numeral 124. Repositioning sheath 126 is configured with a lumen sized to allow passage of at least catheter 102, but otherwise may have any suitable length and construction.

Generally, a blood pump, such as blood pump 101, may initially be inserted into the patient's vasculature via an introducer sheath assembly. Once the blood pump 101 has been inserted into the patient's vasculature, the operator may advance the blood pump 101 to its desired location in the body (e.g., left heart or right heart). As the catheter 102 advances further into the patient's vasculature, the distal end 124 of repositioning sheath 126 may be inserted into the introducer sheath assembly which acts as a conduit for repositioning sheath 126 to enter the patient's vasculature. In some aspects, the introducer sheath may be removed thereafter, for example, in the case of a tear away design, by tearing the introducer along its length. However, in other aspects, where introducer sheath assembly is an expandable design, it may remain in the body, either surrounding some or all of the repositioning sheath 126, or, in cases where the repositioning sheath 126 remains outside of the body, surrounding catheter 102.

Once repositioning sheath 126 has been fully inserted, the operator may secure it to the patient at or near the insertion site using butterfly 130. In that regard, butterfly 130 may be affixed to the patient (e.g., using adhesives or sutures) in order to secure the repositioning sheath 126 and securement device 132 relative to the patient. Thereafter, once the blood pump has been advanced to the desired location within the patient's body, the operator may use securement device 132 to restrict further movement of the catheter 102 and blood pump 101 within the patient. In that regard, securement device 132 may be any device suitable for optionally allowing and restricting movement of catheter 102 therethrough. In one aspect, the specific securement device 132 depicted in FIG. 1C is a Tuohy-Borst type device. However, other suitable securement devices may be used in place of securement device 132.

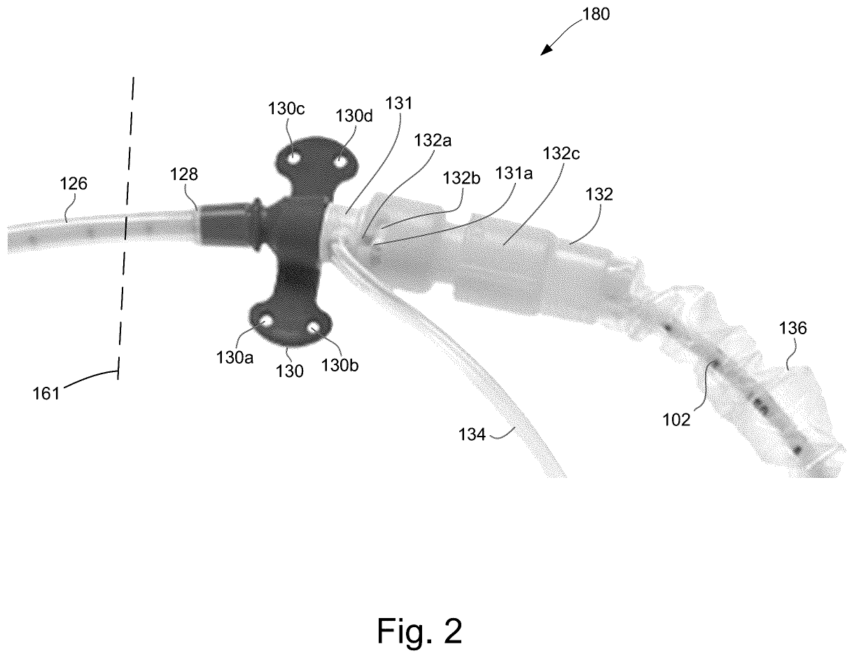

FIG. 2 shows an enlarged view of the blood pump assembly 100 and repositioning sheath assembly 180 of FIG. 1C. FIG. 2 reproduces a portion of blood pump assembly 100 and repositioning sheath assembly 180, oriented with its distal end to the left as though repositioning sheath 126 has been passed through a patient's skin surface (indicated by dashed line 161) from right to left. Oriented in this way, the proximal end 128 of repositioning sheath 126 would remain on the outside of the patient's body, and butterfly 130 may be secured to the patient's skin using sutures passed through suture eyelets 130a, 130b, 130c, and 130d.

The enlarged view of FIG. 2 shows a bayonet connection between hemostasis valve 131 and securement device 132. In that regard, the distal end of securement device 132 is configured such that it can be coupled to a proximal end of hemostasis valve 131 by pushing the two parts together and turning them relative to one another. In the example of FIG. 2, the distal portion of securement device 132 has a cylindrical collar with one or more slots, and the proximal portion of hemostasis valve 131 has a cylindrical projection with one or more pegs 131a. When the cylindrical collar of securement device 132 is advanced over the cylindrical projection of hemostasis valve 131, each peg 131a will enter one of the slots in securement device 132 at an entrance point 132a. Then, by rotating the securement device 132 relative to hemostasis valve 131, each peg 131a will move toward an end point 132b of the slot, thus preventing securement device 132 from being pulled away from hemostasis valve 131 (without first rotating the two components in the opposite direction). In addition, the internal interface between the cylindrical collar of securement device 132 and hemostasis valve 131 may include a deformable seal or gasket (e.g., a rubber washer) to both provide a seal between the parts and to provide backpressure tending to prevent peg 131a from easily moving within the slot of securement device 132. Moreover, a detent may be provided at the end points 132b of each slot in securement device 132 so that peg 131a will tend to remain in the locked state.

As already noted, in the example of FIGS. 1C and 2, the securement device 132 is a Tuohy-Borst device in which a barrel 132c is rotated to vary the amount of resistance imposed on whatever object (e.g., catheter 102) is within the securement device 132. As will be appreciated, other securement devices may be used in other embodiments.

As described herein, the intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. An exemplary implementation of this positioning is shown in FIG. 1D. Once positioned in this way, the intracardiac blood pump assembly 100 may deliver blood from the blood inflow cage 114, which sits inside the left ventricle, through cannula 110, to the blood outflow cage 106, which sits inside the ascending aorta. In some aspects of the technology, intracardiac blood pump assembly 100 may be configured such that bend 118 will rest against a predetermined portion of the patient's vasculature when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

While and/or after the blood pump 101 is inserted into the intended location (e.g., the left ventricle and ascending aorta in the case of a left heart pump) of the patient, the precise orientation and position of blood pump 101 may need to be adjusted to place the inlet 114 and outlet 106 in the desired positions relative to the body structures of the patient. For example, to avoid suction with the tissues and/or surfaces in the left ventricle, it may be desirable for the inlet 114 to be positioned in the free space of the left ventricle such that the inlet 114 does not contact the inner walls of the left ventricle or the chordae that actuate the mitral valve. This position may be achieved by orienting the distal portion 111 of the blood pump 101 toward the apex of the left ventricle and away from the chordae that actuate the mitral valve and having appropriate spacing away from the internal walls of the left ventricle.

In some instances, when the pump 101 is inserted into the patient's heart, the pump 101 may not be in the desired orientation to effectively pump blood and prevent suction events. This may occur for several reasons. For example, prior to being inserted into the patient, the catheter 102 and pump 101 may have an inherent or resting state shape. For purposes herein, the inherent or resting state shape is the shape the catheter 102 and pump 101 take when no external forces or stresses are being applied to the catheter 102 and pump 101. For example, when at rest and exterior to the patient, e.g., placed on a flat surface such as a table, the pump 101 and catheter 102 may be disposed in the same two-dimensional plane (e.g., in this case, defined by the surface of the table). Due to the inherent resting state shape of the catheter 102 and pump 101, when the pump 101 is inserted into the patient's heart, the pump 101 may not be in the desired orientation or position and thus may need to be adjusted to achieve the desired position. For example, the resting state shape of the catheter 102 and pump 101 may cause the distal portion 111 of the pump, including the inlet 114, to initially be biased toward and in contact with the ventricular walls or other structures in the left ventricle, which may cause the inlet 114 to be blocked or reduce its capacity to draw in blood into the cannula 110. As will be further appreciated, due to movement of the patient and/or movement of the pump during use, the position of the pump may need to be adjusted for continued use.

In some instances, to alter the position of the pump 101 within the patient, a user may attempt to grasp the portion of the catheter 102 that is exterior to the patient and apply torque to the catheter 102 to torsion/twist the catheter 102 and thereby rotate the cannula 110 to change the orientation of distal portion 111 of pump 101 within the patient's heart. Moreover, the user may pull (proximally) or push (distally) the catheter 102 into or out of the patient. Referring to FIG. 1C, the portion of the catheter 102 that is exterior to the patient when the pump 101 is inserted in the patient is indicated by reference numeral 181. Portion 181 is the portion of catheter 102 that is distal of plug 138 and proximal of fixation device or butterfly 130 when butterfly 130 is affixed to the patient proximate to the access site. Some or all of portion 181 may be covered by protection sleeve 136 and/or a securement device, such as securement devices 132.

However, the user may find that, when they grip and torque portion 181 of the catheter 102, the catheter 102 may not efficiently transmit or transfer the torque from the area the catheter 102 is gripped to the distal end of the catheter 102. Thus, the catheter 102 may transmit a significantly lower amount of torque to the distal end of the catheter 102 than the torque applied to portion 181 of catheter 102 by the user. Efficient transfer of torque to the distal end of catheter 102 is sought for several reasons. For example, when some or all of the torque applied to portion 181 of catheter 102 is not transmitted to the distal end of the catheter 102, the distal end of catheter 102 and thus the pump 101 may be rotationally displaced less (or not at all) relative to the portion of the catheter 102 gripped by the user. This may make adjustments to the orientation of pump 101 inaccurate and/or may makes the user uncertain of what the current orientation of the pump 101 is within the patient. Additionally, less than efficient torque transfer may lead the user to apply excessive torque to the catheter 102 when attempting to rotate pump 101. However, the torque that is not initially transferred to the distal end of the catheter 102, may be stored (e.g., in the form of torsional deformation) in the catheter 102 and may then be released at a later time (e.g., partially and/or all at once) in an unintended manner, which could cause the pump 101 to recoil and end up in an undesirable position.

In addition to the above-described challenges, the user may also find that it is difficult to apply torque to catheter 102. In this regard, the proximal end of catheter 102 is coupled to several components of assembly 100. For example, as described above, in some aspects, the proximal end of catheter 102 may be coupled to plug 138. Plug 138 may be further coupled to cable 160, tubing 155, and controller 142. Thus, when the user grips and attempts to torque a portion of catheter 102 that is exterior to the patient, the plug 138 and other components of assembly 100 proximal to catheter 102 may resist the user's attempt to apply torque to catheter 102. This may further inhibit the user's ability to torque catheter 102 to steer and position pump 101 within the patient as desired.

Described herein are structures and methods that improve the torque transmission characteristics of catheters for catheter assemblies. For example, the structures and methods described herein may improve the torque transmission characteristics of catheters, such as, catheter 102 in order to steer pump 101 within the patient. The present technology provides a catheter for a catheter assembly including a reinforcement structure embedded in a wall or layer of the catheter. The reinforcement structure may be configured such that a distal portion of the catheter transmits torque toward the distal end of the catheter within acceptable levels of efficiency. Moreover, the reinforcement structure may be configured such that a proximal portion of the catheter transmits torque less efficiently (i.e., reduces torque transmission) toward the proximal end of the catheter. In this way, the distal portion of the catheter including the reinforcement structure in accordance with the present technology may improve the user's ability to accurately torque the catheter to thereby torque and steer the pump. Moreover, the proximal portion of the catheter including the reinforcement structure in accordance with the present technology may reduce resistance to torquing the catheter.

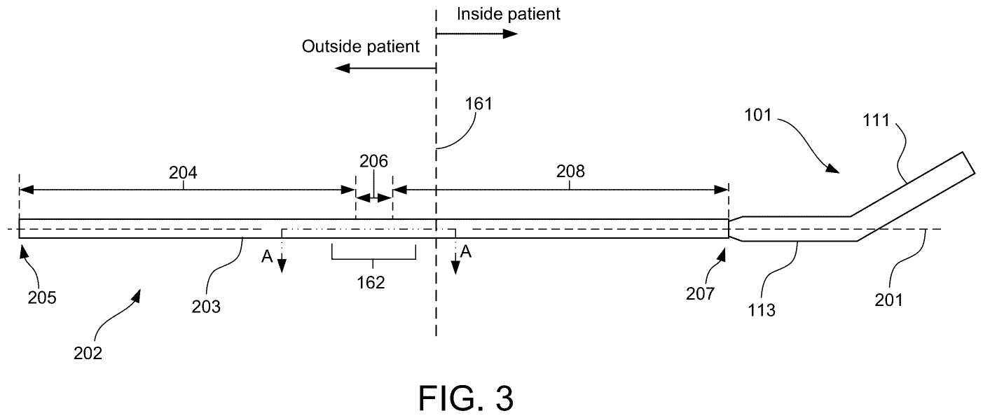

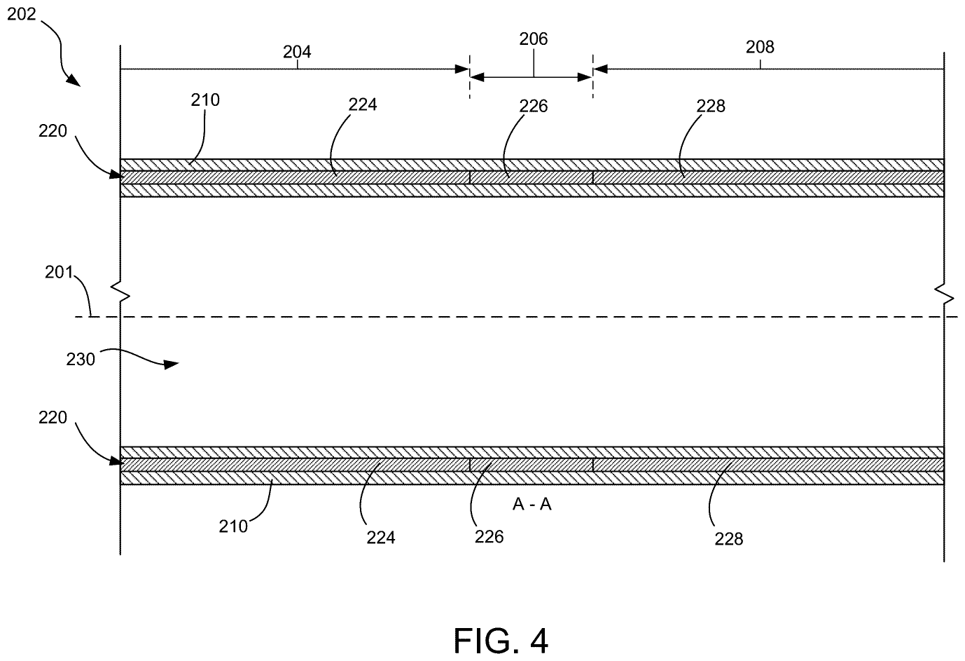

For example, referring to FIGS. 3 and 4, a catheter 202 is shown coupled to blood pump 101 for use with assembly 100 in accordance with aspects of the present technology. As will be described in greater detail, catheter 202 may include a reinforcement structure 220 (see FIG. 4) in accordance with the present technology for improving the torque transmission characteristics of catheter 202. It is to be appreciated that catheter 202 may be used with a catheter assembly, such as an intracardiac catheter assembly, or other types of catheter assemblies inserted into a patient. Moreover, it is to be appreciated that catheter 202 is not shown to scale in FIGS. 3 and 4.

As shown, catheter 202 comprises an elongate tubular body 203 extending along a longitudinal axis 201 from a proximal end 205 to a distal end 207 of body 203. In one aspect, proximal end 205 is coupled to plug or handle 138 and distal end 207 is coupled to the proximal end (e.g., to proximal portion 113) of blood pump 101. The tubular body 203 includes at least one lumen 230 extending from proximal end 205 to distal end 207 along axis 201. It is to be appreciated that, in some aspects, catheter 202 may include a plurality of lumens extending between proximal end 205 and distal end 207 to carry electrical wires, purge lines, optical fibers, and/or other wires/lines of assembly 100.

Body 203 comprises a layer 210 forming the jacket of body 203. In one aspect, layer 210 forms the inner and outer surfaces of body 203. The layer 210 may be made of a polymer, such as polyurethane. Body 203 further comprises a reinforcement structure 220 embedded in layer 210. In one aspect, layer 210 may encapsulate reinforcement structure 220. Reinforcement structure 220 extends within layer 210 along longitudinal axis 201 from the proximal end 205 to the distal end 207 of body 203. In one aspect, reinforcement structure 220 may have an elongate tubular shape and is coaxially arranged with layer 210 along axis 201. Reinforcement structure 220 may be made of a metallic material. In some aspects, reinforcement structure 220 is made of a metal alloy, such as nitinol. Body 203 with reinforcement structure 220 may be configured to be sufficiently flexible to permit catheter 202 to bend within the vasculature of a patient.

Body 203 may comprise a proximal portion 204 and a distal portion 208. As will be described in greater detail, the reinforcement structure 220 may be configured such that the proximal portion 204 of body 203 has different torque transmission characteristics than the distal portion 208 of body 203.

As shown in FIG. 4, the reinforcement structure 220 may include a proximal portion 224 and a distal portion 228. The proximal portion 224 extends within layer 210 along axis 201 from the proximal end to the distal end of proximal portion 204 of body 203. The distal portion 228 may extend within layer 210 along axis 201 from the proximal end to the distal end (i.e., distal end 207) of distal portion 208 of body 203.

Proximal portion 224 of reinforcement structure 220 may be configured to reduce the amount of torque transmitted from one end of proximal portion 204 of body 203 to an opposite end of proximal portion 204. In one aspect, proximal portion 224 is configured as a spiral or coil (e.g., a nitinol coil) that is wound around axis 201. The coil portion 224 of reinforcement structure 220 may provide kink resistance to portion 204 of body 203, while at the same time maintaining flexibility. Furthermore, the coil portion 224 may be configured such that, proximal portion 204 transmits torque inefficiently over the length of portion 204. In one aspect, proximal portion 204 is configured to transmit 1-20% of the torque inputted at the distal end of proximal portion 204 to the proximal end of portion 204. Thus, a large rotational displacement at the distal end of portion 204 (e.g., due to a user torquing catheter 202) will produce a much smaller rotational displacement at the proximal end of portion 204 that is coupled to plug or handle 138. Accordingly, the inefficient torque transmission of portion 204 of body 203 reduces the amount of resistance a user may experience when applying torque to catheter 202.

Distal portion 228 of reinforcement structure 220 may be configured such that distal portion 208 of body 203 transmits a large proportion (e.g., 50%-100%) of torque inputted at one end of distal portion 208 of body 203 to an opposite end of distal portion 208. Thus, distal portion 208 may be configured to transfer a large proportion (e.g., 50%-100%) of the rotational displacement at the proximal end of portion 208 (e.g., due to a user torquing catheter 202) to the distal end 207 of portion 208. Accordingly, portion 208 of body 203 may reduce the chances of catheter 202 recoiling after being torqued by a user. Moreover, portion 208 may permit more accurate torquing of pump 101.

In one aspect, distal portion 228 of reinforcement structure 220 may be formed by laser cutting the material of structure 220 (e.g., nitinol) according to a predetermined pattern. The predetermined pattern is selected to impart the desired torque transmission characteristics to distal portion 208. For example, the predetermined pattern is selected such that distal portion 208 transmits torque more efficiently than portion 204 of body 203. Moreover, the predetermined pattern is selected such that distal portion 208 is sufficiently flexible to permit insertion and navigation of catheter 202 into the vasculature of the patient.

In one aspect, the predetermined laser cut pattern comprises a lattice structure.

In one aspect, the predetermined laser cut pattern comprises a plurality of apertures or windows in portion 228 of reinforcement structure 220. The apertures may be arranged to form superimposed torque paths along the material of structure 220 between the apertures. In one aspect, a first torque path and a second torque path may be formed by the material of the reinforcement structure 220 that is between the apertures. The first torque path and the second torque path may each be spirals that are wound around axis 201. The first spiral torque path may be wound around axis 201 in an opposite direction to the second spiral torque path. The first and second torque paths may enable portion 208 of body 203 to efficiently transmit torque both when catheter 202 is torqued clockwise about axis 201 and when catheter 202 is torqued counterclockwise about axis 201. Thus, the predetermined laser cut pattern facilitates bi-directional torque transmission by having a continuous pathway for torsional load to be delivered in opposing directions.

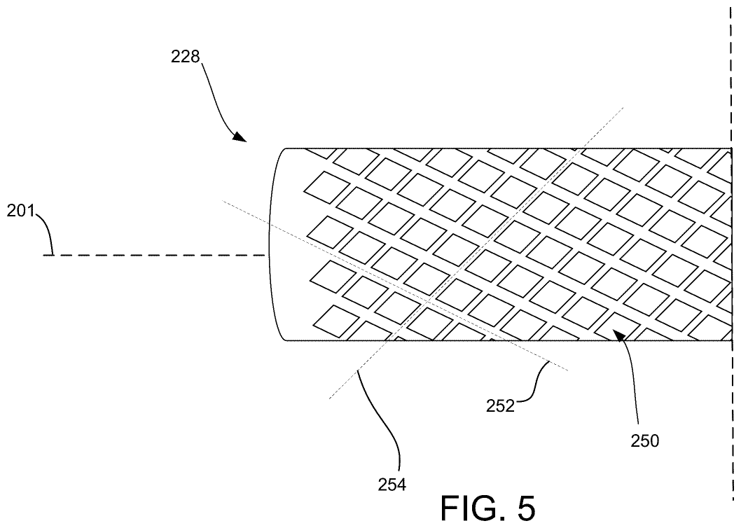

An exemplary implementation of the predetermined laser cut pattern according to aspects described herein is shown in FIG. 5. As shown in FIG. 5, reinforcement structure 220 may be laser cut according to a pattern comprising apertures 250 in the material of reinforcement structure 220 in portion 228. The apertures 250 may have a predetermined shape and a predetermined spacing/distribution according to the pattern. The apertures 250 may be diamond-shaped as shown or may have another shape (rectilinear, curved, etc.). At least a first torque path 252 and a second torque path 254 is formed along the material between apertures 250. Torque paths 252 and 254 may be superimposed spirals that are wound around axis 201 in opposite directions. The shape of each of the apertures 250 and the spacing between apertures 250 are properties that, among others, define the torque transfer characteristics and torque paths of portion 228 of reinforcement structure 220.

In one aspect, the predetermined laser cut pattern of portion 228 may include elements or features configured to enhance the flexibility of portion 208 of body 203 and minimize length changes under torsional load.

The inventors have recognized that the structure of portion 228 resulting from the predetermined laser cut pattern may provide several advantages and benefits when implemented in a catheter as described herein. For example, portion 228 of reinforcement structure 220 of the present technology can achieve efficient bi-directional torque transmission (i.e., when rotated in either direction) in a single layer by providing the above-described superimposed torque paths. Thus, the overall wall thickness of catheter 202 may be decreased relative to reinforcement structures including multiple concentric layers.

In one aspect, body 203 further includes an intermediate portion 206 disposed between proximal portion 204 and distal portion 208. Moreover, reinforcement structure 220 may further include an intermediate portion 226 disposed between proximal portion 224 and distal portion 228. Intermediate portion 226 extends within layer 210 along longitudinal axis 201 from a proximal end of intermediate portion 206 of body 203 to a distal end of intermediate portion 206 of body 203. Intermediate portion 226 may be configured to couple proximal portion 224 to distal portion 228. Moreover, intermediate portion 226 may comprise geometric features configured to promote mechanical connections between intermediate portion 226 and the layer 210. For example, the geometric features may comprise protrusions extending from an outer surface of portion 226 toward the exterior of body 203. The geometric features may additionally or alternatively comprise apertures in the material (e.g., nitinol) of which the reinforcement structure 220 is made. In one aspect, the material of layer 210 (e.g., a polymer) may be reflowed onto reinforcement structure 220 to form catheter 202. In this aspect, the geometric features of intermediate portion 226 promote mechanical interlocking between the geometric features of portion 226 and the material of layer 210. In this way, intermediate portion 226 promotes a secure mechanical connection between reinforcement structure 220 and layer 210.

It is to be appreciated that, in some aspects, reinforcement structure 220 may be bonded chemically to layer 210. The chemical bond between reinforcement structure 220 and layer 210 may be in addition to the mechanical connections (e.g., mechanical interlocking) between reinforcement structure 220 and layer 210. Alternatively, the chemical bond between reinforcement structure 220 and layer 210 may replace the mechanical connections.

Portions 204, 206, and 208 of body 203 may each have predetermined longitudinal lengths selected to appropriately position portions 204, 206, and 208 in relation to the patient to take advantage of the different torque transmission characteristics of portion 204 and 208 of body 203. For example, the patient's skin at the access site for the pump assembly is indicated in FIG. 3 by dotted line 161, which forms a boundary between the exterior of the patient and the interior of the patient. As shown, proximal portion 204 of body 203 and proximal portion 224 of reinforcement structure 220 may be disposed exterior to the patient when the distal portion of catheter 202 and the pump 101 are inserted into the patient. Intermediate portion 206 of body 203 and intermediate portion 226 of reinforcement structure 220 are also disposed exterior to the patient when the distal portion of the catheter 202 and pump 101 are inserted into the patient. Described another way, distal portion 208 of body 203 and the distal portion 228 of reinforcement structure 220 may be predominantly disposed within the patient. As shown, in one aspect, a small length (e.g., 1% to 10% of the total length) of distal portion 208 of body 203 and distal portion 228 of reinforcement structure 220 is disposed exterior to the patient, with the remainder being introduced into the vasculature of the patient.

As described above, after a catheter of a pump assembly, such as catheter 202, is inserted into a patient, a user may grip the portion of catheter 202 that is exterior to the patient and apply torque to catheter 202 to steer and position pump 101 to a desired position and orientation within the patient. Typically, a user will grip a portion of the catheter 202 that is exterior to the patient and near the access site (i.e., near dotted line 161 in FIG. 3). Where a securement device, such as securement device 132 is used, a user will grip the catheter 202 near the proximal end of securement device 132. Thus, the user may grip catheter 202 over intermediate portion 206 of body 203 or at least near intermediate portion 206, such as at the distal end of portion 204 or at the proximal end of portion 208. It is to be appreciated that the user's grip may span one or more of the intermediate portion 206, the distal end of portion 204, and/or the proximal end of portion 208. For example, in FIG. 3, the area 162 of body 203 is indicated over which a user may grip and apply torque to catheter 202.

When a user applies torque to catheter 202 over area 162, proximal portion 204 and distal portion 208 of body 203 will each transmit the torque away from area 162 with differing levels of efficiency. For example, due to the torque transmission characteristics of proximal portion 224 of reinforcement structure 220, proximal portion 204 is configured to transmit a first proportion or ratio of the externally applied torque from the distal end to the proximal end of portion 204. Moreover, due to the torque transmission characteristics of distal portion 228 of reinforcement structure 220, distal portion 208 may be configured to transmit a second proportion or ratio of the externally applied torque from the proximal end to the distal end of portion 208 (i.e., distal end 207). The second proportion of torque transmitted by portion 208 may be larger than the first proportion of torque transmitted by portion 204. In one aspect, distal portion 208 is configured to transmit 50 to 100% of the torque inputted at the proximal end of portion 208 to the distal end (i.e., end 207) of portion 208. In one aspect, proximal portion 204 may be configured to transmit 1% to 20% of the torque inputted at the distal end of proximal portion 204 to the proximal end of portion 204, although the proximal portion may be configured to transmit other suitable percentages or torques.

In one aspect, portions 224, 226, and 228 of reinforcement structure 220 are interconnected to each other and formed during manufacturing starting from a single tube made of a suitable material (e.g., nitinol) for the reinforcement structure.

It is to be appreciated that in other aspects of the present technology, one or both of portions 224 and 228 of reinforcement structure 220 may have a different structure or configuration than described above.

For example, in one aspect, the proximal portion 224 may be configured as described above as a spiral or coil (e.g., a nitinol coil) that is wound around axis 201 in a first direction. However, in this aspect, in contrast to the aspects described above, distal portion 228 may also be configured as a spiral or coil structure (e.g., made of nitinol) that is configured to transfer torque more efficiently than proximal portion 224. For instance, in one example of this aspect, the distal portion 228 may be a spiral or coil (e.g., a nitinol coil) that has a larger gauge winding and/or a different pitch relative to the spiral or coil of portion 224 such that the distal portion 228 transfers torque more efficiently than the proximal portion 224. Additionally, or alternatively, the distal portion 228 may be configured as a multi-layered concentric coil structure, similar to a torque cable. For example, the distal portion 228 may comprise two, three, or more distinct, concentric layers of spirally wound coils, which are wound in alternating directions in each successive layer. Such a multi-layered coil structure transfers torque more efficiently than the single-layered coil structure used in the proximal portion 224.

It is to be appreciated that there are many different combinations of structures/configurations that may be used for the proximal portion 224 and distal portion 228 of the reinforcement structure 220 described herein. However, in all aspects of the present technology, the reinforcement structure 220 is configured such that the proximal portion 224 has different torque transfer characteristics relative to the distal portion 228 such that the distal portion 228 transfers torque more efficiently than the proximal portion 224 of the reinforcement structure 220.

It is to be appreciated that the term “efficiency” as used herein in relation to torque transfer or transmission, describes the proportion, ratio, or amount of externally applied torque that is transferred or transmitted by a region, portion, or length of an elongate structure (such as a catheter, a tube, or other elongate structure) from a first location of the section/region (where the torque is externally applied/inputted) to a second location of the section/region (e.g., distal or proximal of the first location). The “efficiency” may be characterized in terms of a comparison (percentage, ratio, difference) of the inputted torque at the first location, which is externally applied by the user, relative to the torque as measured at the second location. Thus, statements herein regarding a section/region of an elongate structure (e.g., a catheter) that transfers or transmits torque more/less efficiently than another section/region of the elongate structure refer to the differences in the proportion, ratio, or amount of torque that each section of the elongate structure is configured to transmit over its length.

From the foregoing and with reference to the various figure drawings, those skilled in the art will appreciate that certain modifications may also be made to the present disclosure without departing from the scope of the same. While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1. A catheter for a catheter assembly, the catheter comprising:
an elongate tubular body comprising:
a proximal end and a distal end,
at least one lumen extending from the proximal end to the distal end along a longitudinal axis,
a layer made of a first material,
a first portion having a first length,
a second portion having a second length, wherein the first portion is proximal of the second portion, and
a reinforcement structure embedded in the layer,
wherein the reinforcement structure is configured such that the second portion of the elongate tubular body transmits a larger proportion of externally applied torque over the second length than the first portion transmits over the first length.

2. The catheter of claim 1, wherein the reinforcement structure is made of a second material.

3. The catheter of claim 2, wherein the first material is a polymer.

4. The catheter of claim 2, wherein the first material is a polyurethane.

5. The catheter of claim 2, wherein the second material is a metal alloy.

6. The catheter of claim 5, wherein the second material is nitinol.

7. The catheter of claim 1, wherein the reinforcement structure comprises a first portion that extends from a first end to a second end of the first portion of the elongate tubular body, and the first portion of the reinforcement structure is configured as a coil.

8. The catheter of claim 7, wherein the reinforcement structure comprises a second portion that extends from a first end to a second end of the second portion of the elongate tubular body, and the second portion of the reinforcement structure is laser cut according to a predetermined pattern.

9. The catheter of claim 8, wherein the predetermined pattern comprises apertures in the reinforcement structure that are arranged such that a first spiral torque path and a second spiral torque path are formed by material of the reinforcement structure between the apertures.

10. The catheter of claim 9, wherein the first spiral torque path and second spiral torque path are wound about the longitudinal axis in opposing directions.

11. The catheter of claim 9, wherein the apertures are each diamond-shaped.

12. The catheter of claim 8, wherein the predetermined pattern comprises a lattice structure.

13. The catheter of claim 1, wherein the reinforcement structure has an elongate tubular shape.

14. The catheter of claim 1, wherein the reinforcement structure is coaxial with the elongate tubular body.

15. The catheter of claim 1, wherein the elongate tubular body further comprises an intermediate portion disposed between the first portion and the second portion and the reinforcement structure further comprises an intermediate portion that extends from a first end to a second end of the intermediate portion of the elongate tubular body.

16. The catheter of claim 15, wherein the elongate tubular body is formed by reflowing the first material onto the reinforcement structure and the intermediate portion of the reinforcement structure includes one or more features configured to promote mechanical interlocking between the one or more features and the layer when the first material is reflowed onto the reinforcement structure.

17. A intracardiac blood pump assembly, comprising:
a blood pump comprising a proximal portion and a distal portion; and
a catheter comprising an elongate tubular body, the elongate tubular body comprising:
a proximal end and a distal end, wherein the distal end of the elongate tubular body is coupled to the proximal portion of the blood pump,
at least one lumen extending from the proximal end to the distal end along a longitudinal axis,
a layer made of a first material,
a first portion having a first length,
a second portion having a second length, wherein the first portion is proximal of the second portion, and
a reinforcement structure embedded in the layer,
wherein the reinforcement structure is configured such that the second portion of the elongate tubular body transmits a larger proportion of externally applied torque over the second length than the first portion transmits over the first length.

18. The intracardiac blood pump assembly of claim 17, wherein the reinforcement structure comprises a first portion that extends from a first end to a second end of the first portion of the elongate tubular body, and the first portion of the reinforcement structure is configured as a coil.

19. The intracardiac blood pump assembly of claim 18, wherein the reinforcement structure comprises a second portion that extends from a first end to a second end of the second portion of the elongate tubular body, and the second portion of the reinforcement structure is laser cut according to a predetermined pattern.

20. The intracardiac blood pump assembly of claim 19, wherein the predetermined pattern comprises apertures in the reinforcement structure that are arranged such that a first spiral torque path and a second spiral torque path are formed by material of the reinforcement structure between the apertures, wherein the first spiral torque path and second spiral torque path are wound about the longitudinal axis in opposing directions.
